(19) United States
(12) Patent Application Publication
(10) Pub. No.: US 2006/0135934 A1
(43) Pub. Date: Jun. 22, 2006

(54) Feminine hygiene product

(76) Inventor: Tawanda F. Gilbert, Oakland, CA (US)

Correspondence Address: KIRKPATRICK & LOCKHART NICHOLSON GRAHAM LLP, 535 SMITHFIELD STREET, PITTSBURGH, PA, 15222, US

(21) Appl. No.: 11/016,276

(22) Filed: Dec. 17, 2004

(51) Int. Cl. A61F 13/15

(52) U.S. Cl. 604/385.101; 604/385.17; 604/385.04

(57) ABSTRACT

Absorbent articles for absorbing a bodily fluid comprise a pad region and a pre-formed conformable gluteal region. The pad region includes a first absorbent core, a liquid pervious surface, and a liquid impervious surface, wherein the absorbent core of the pad region is positioned between the liquid pervious surface and the liquid impervious surface. The conformable gluteal region includes a second absorbent core that is in fluid connection with the first absorbent core in the pad region. The gluteal region is configured to secure at least a portion of the absorbent article between a user's buttocks, and is further configured to wick fluid from the pad region. The absorbent article extends greater than about 12 inches in length.

### FIELD OF THE INVENTION

The described devices relate to extended fluid absorption articles, such as sanitary napkins or incontinence pads with exceptional absorbency and coverage. These devices particularly relate to pads conformed to secure between the buttocks by an extended region which also acts to hold fluid.

### BACKGROUND

Absorbent hygienic pads (for example, without limitation, sanitary napkins, pantyliners, disposable diapers, bandages, and the like) are intended to absorb and retain liquid discharges from the human body such that the liquid is prevented from extended contact with the user, and from soiling the user's clothing. However, many hygienic pads cover only a limited region of the perineal area in most women. Further, many hygienic pads have a limited absorbency, and may therefore leak and become uncomfortable. Finally, smaller pads may shift position as a woman moves, even if secured to undergarments.

One of the difficulties in designing a pad to overcome these problems has been that women have an almost infinite variety of body shapes and muscle tone in the upper thigh and buttock region, and therefore pads, which provide comfort and protection for some women, may be deficient when worn by women of different body shapes. This problem is particularly acute for women with larger body sizes.

It has also been recognized that a certain percentage of women have extremely heavy flow during portions of the menstrual cycle. Existing absorbent pads may be inadequate for such women. In particular, most women have heavy menstrual discharge early in the morning due to the retention of menses during sleep followed by a sudden discharge upon awakening and rising.

U.S. Pat. No. 4,597,759 to Johnson describes a hygienic pad such as a sanitary napkin having extensions that can be attached to a central pad unit to increase the dimension in a direction determined by the user.

Likewise, US Patent application 2003/0195488 by Van Gompel et. al., describes an elongated absorbent pad, normally curved upward and having opposite longitudinal elasticized raised side edges which are deflectable with respect to a central portion of the pad. The pad includes an absorbent contained between a liquid-permeable cover and a liquid-impermeable baffle and may include a tension mechanism for inducing the upward curve of the pad. The pad also contains a control means secured adjacent to the side edges for selectively controlling the deflection of at least one of the side edges during use so as to optimize the function of the sanitary napkin. The control means are anchorable under the preventing the side edges from folding in toward the absorbent.

U.S. Pat. No. 5,743,896 describes an sanitary napkin including a first padding section having a channel that is formed along the longitudinal axis thereof; a second padding section sized in a manner such that an extending portion of the second padding section extends away from the second surface of the first padding section when a portion of the second padding section is disposed within the channel; a screen barrier having a screen channel formed within the screen barrier at a location allowing the extending portion of the second padding section to be entirely disposed within the screen channel when the screen barrier is placed over the surface of the first padding section; and a liquid impermeable barrier section entirely covering the back surface of the first padding member and extending over the perimeter of the screen barrier and adhesively secured to the exterior surface of the screen barrier in a manner to seal the first and second padding sections between the liquid impermeable section and the screen barrier.

EP Patent number 0136524 by Nakano et al., describes a sanitary napkin including at least an absorbent material for menstrual blood, a blood-impermeable material and a covering material, characterized in that the sanitary napkin is provided with at least one bending groove which, during use, can produce a configuration conforming to the shape of the perineal area of the wearer.

U.S. Pat. No. 6,316,688 to Hammons et al., describes a sanitary napkin that has a three dimensionally-shaped tube of absorbent material and a method of making it. The sanitary napkin comprises a base pad having a body-facing side, a garment-facing side, and a longitudinal center line. A tube of absorbent material extends outward from the body-facing side of the base pad and is aligned along the longitudinal centerline of the base pad.

None of the cited references shows or suggests the present invention as described herein. There is a need for an absorbent article capable of absorbing an extremely heavy flow of menstrual fluid. Further, there is a need for absorbent pads having an extended shape configured to both secure the article between the user's buttocks and to absorb bodily fluids.

### SUMMARY OF INVENTION

Described herein are absorbent articles for absorbing a bodily fluid. In a particular embodiment, the absorbent articles comprise a pad region and a non-corrugated conformable gluteal region. The pad region comprises a first absorbent core, a liquid pervious surface, and a liquid impervious surface, wherein the absorbent core of the pad region is positioned between the liquid pervious surface and the liquid impervious surface. The non-corrugated, conformable gluteal region comprises a second absorbent core that is in fluid connection with the first absorbent core in the pad region. The gluteal region is configured to secure at least a portion of the absorbent article between a user's buttocks. The gluteal region is further configured to wick fluid from the pad region. The absorbent article also extends at least about 12 inches in length.

In another embedment, the first absorbent core of the absorbent article may comprise a non-woven material. In yet another embodiment, the first absorbent core comprises cotton.

In a further embodiment, the absorbent article further comprises at least one fastener adapted to attach to a garment. In a particular embodiment, the fastener comprises adhesive tape. In another particular embodiment, the fastener comprises wings. In an alternative embodiment, the fastener does not include wings. In yet another embodiment, at least one fastener is located on the liquid impervious surface of the pad region. In another alternative embodiment, the fastener is attached to the gluteal region. In a further embodiment, the fastener adheres to the skin of the user.

In a particular embodiment, the absorbent article further comprises a deodorant. In another embodiment, the absorbent article further comprises an antibacterial agent. In yet another embodiment, the absorbent article's gluteal region further comprises a flexible gluteal insert region. In a particular embodiment, the gluteal insert region is a cylinder.

In another embodiment, the absorbent article comprises a gluteal region that is tapered. In yet another embodiment, the width of the gluteal region is narrower than the width of the pad region.

In other embodiments, the absorbent article substantially conforms to a female perineal body region and is configured to absorb menstrual fluid. In another embodiment, the absorbent article is foldable.

In an embodiment, an absorbent article for absorbing a bodily fluid from a user comprises a pad region and a gluteal region. The pad region of this particular embodiment comprises a liquid pervious surface, a liquid impervious surface, and a first region of an absorbent core positioned between the liquid pervious and liquid impervious surfaces. This embodiment also includes a gluteal region that comprises a second region of the absorbent core, wherein at least a portion of the gluteal region provides stability for the absorbent article when worn by a user. This embodiment also further comprises a gluteal insert region and the absorbent article extends at least about 12 inches in length.

In another embodiment, an absorbent article for absorbing a bodily fluid from a user comprises a pad region and a gluteal region. The pad region of this particular embodiment comprises a liquid pervious surface, a liquid impervious surface and a first region of an absorbent core positioned between the liquid pervious and liquid impervious surfaces. The gluteal region of this particular embodiment further comprises a second region of the absorbent core, wherein at least a portion of the gluteal region provides positional stability for the absorbent article when worn by the user. This particular embodiment also includes a flexible gluteal insert region, wherein the gluteal region is configured to wick fluid from the pad region. This particular embodiment further includes that the absorbent article extends at least about 12 inches in length.

### BRIEF DESCRIPTION OF THE DRAWINGS

FIG. 1A is a proximal view of one embodiment of the absorbent article described herein.

FIG. 1B is a cross-sectional view of the absorbent article of FIG. 1A taken through line B-B′ showing a cross-section of a pad region.

FIG. 1C is a cross-sectional view of the absorbent article of FIG. 1A taken through line C-C′, showing a cross-section of the gluteal region.

FIG. 2A is a proximal view of another embodiment of the absorbent article described herein.

FIG. 2B is a lateral view of the absorbent article shown in FIG. 2A.

FIG. 2C is a distal view of the absorbent article shown in FIG. 2A with an additional gluteal region fastener and wings.

FIGS. 3A to 3C show alternative embodiments of the absorbent article described herein.

FIG. 4A is a proximal view of another embodiment of the absorbent article described herein.

FIG. 4B to 4D show alternative cross-sectional embodiments of the gluteal region of the absorbent article of FIG. 4A taken though line D-D′.

FIG. 5 shows a distal view of one embodiment of the absorbent article being worn by a user.

FIG. 6 shows a cut-away view of the absorbent article being worn by a user as in FIG. 5.

### DETAILED DESCRIPTION

The devices and methods of use and manufacture described herein relate to extended absorbent articles for absorbing bodily fluids comprising a pad region, a gluteal region and an absorbent core throughout, wherein the absorbent core of the pad region is in fluid connection with the absorbent core of the gluteal region.

As used herein, the term “absorbent article” or “absorbent articles” refers to an article to be worn by a person in need thereof (usually a woman) in order to absorb bodily emissions. Absorbent articles may also be referred to as sanitary napkins, hygienic pads, etc. In general, the absorbent articles described herein are configured to be worn adjacent to the pudendal/perineal region of a user, extending posteriorly towards the coccyx or tailbone region.

The absorbent articles described herein are configured to absorb and contain bodily secretions emitted in this region. Any bodily fluid or secretion may be absorbed by these devices. For example, menses, blood, urine, sweat, etc. The absorbent article may absorb all of these fluids equivalently, or one or more preferentially. The absorbent articles described herein may be useful for absorbing extremely heavy fluid flows, particularly heavy menstrual flows. The extended gluteal region, which may tuck between the user's buttocks, in particular embodiments, acts to “wick” or remove excess fluid from the pad region adjacent to the user's genitourinary region. Thus, the absorbent articles substantially conform to the body shape of a user and thereby prevent leakage and excessive shifting (or other movement) of the absorbent article when worn by a user. In particular, the device may be sized so as to fit users having different body sizes and shapes. As used herein, the term “proximal” means towards the body of the user. It is intended that a proximal surface of the device is a body-facing surface when the device is in use by a user. Also as used herein, the term “distal” means away from the body of the user. It is intended that a distal surface of the device is either a surface facing away from the user or is a garment-facing surface when the device is in use by a user.

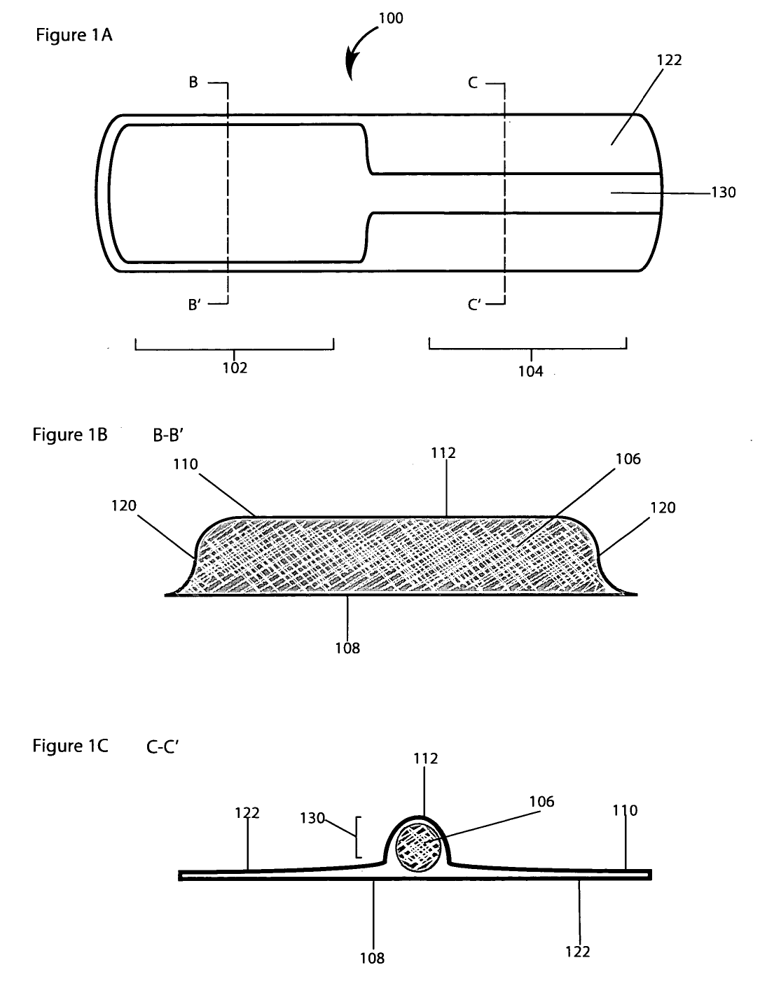

Examples of absorbent article devices 100 are shown in FIGS. 1 to 6. FIG. 1 shows the three general regions of the absorbent article 100: pad region 102, gluteal region 104, and absorbent core 106. Each of these regions is discussed in detail below. At least a portion of pad region 102 is configured to have a liquid impervious surface on distal surface 108 (garment facing surface) of absorbent article 100, and another region is configured to have a liquid pervious surface 112 on proximal surface 110 (body facing surface). Absorbent core 106 in pad region 102 is generally located between theses two surfaces 110, 108. Absorbent core 106 of pad region 102 is in fluid communication with absorbent core 106 of gluteal insert 130.

Although FIGS. 1-4 illustrate absorbent article 100 in a flattened, fully extended position, it should be understood that absorbent article 100 is generally flexible and will conform to the body shape. In some embodiments, absorbent article 100 has a generally curved shape, (for example, without limitation, a crescent shape) which more closely approximates the curvature of the body region extending from the pudendal region to the tailbone region. In other embodiments, at least pad region 102 of absorbent article device 100 has an expandable edge to allow closer conformity to a user's body. In one embodiment, the edge of at least a region of the absorbent article 100 further comprises elastic.

Pad Region

FIG. 1A shows a proximal view of pad region 102. For the sake of consistency, proximal refers to the body-facing side of absorbent article 100. That is, the portion of absorbent article 100 configured to be placed adjacent to the user's body during use. At least a portion of proximal surface 110 of pad region 102 comprises a liquid pervious surface 112. See FIG. 1B.

The liquid pervious surface 112 located on proximal surface 110 allows fluid (for example, without limitation, menses, blood, urine, etc.) to pass into absorbent article 100 from the body, where it may be absorbed by absorbent core 106 of pad region 102 of absorbent article 100. In some embodiments, liquid pervious surface 112 of proximal surface 110 allows fluid to pass into absorbent core 106, but does not allow fluid to remain on proximal surface 110, thereby keeping proximal surface 110 (the body-contacting surface) relatively dry. Thus, the liquid pervious surface 112 of proximal surface 110 may comprise a porous surface. In one embodiment, liquid pervious surface 112 is a primarily hydrophobic surface having pores that allow fluid to pass into the absorbent core.

The liquid pervious surface 112 may comprise any appropriate material. For example, without limitation, the liquid pervious surface 112 may be made from woven and/or non-woven materials; polymeric materials such as aperture formed thermoplastic films, apertured or unapertured plastic film, and hydroformed thermoplastic films; porous foams, reticulated foams, reticulated thermoplastic films, and thermoplastic scrims. Suitable woven and non-woven materials can comprise natural fibers (for example, without limitation wood or cotton fibers), synthetic fibers (for example, without limitation, polymeric fibers such as polyester, polypropylene or polyethylene fibers); bicomponent fibers (that is fibers having a core of one material which is enclosed in a sheath made of another material), or from a combination of natural and synthetic fibers. In one embodiment, liquid pervious surface 112 is a coating (for example, without limitation, a coating applied directly to the absorbent core).

In an embodiment, liquid pervious surface 112 of proximal surface 110 comprises a flexible surface such as a thin film of apertured material. In another embodiment, liquid pervious surface 112 is flexible. In yet another embodiment, liquid pervious surface 112 comprises a textured pattern. In a further embodiment, liquid pervious surface 112 is comprised of a material further sufficient to contain the absorbent core material 106 within the absorbent article 100.

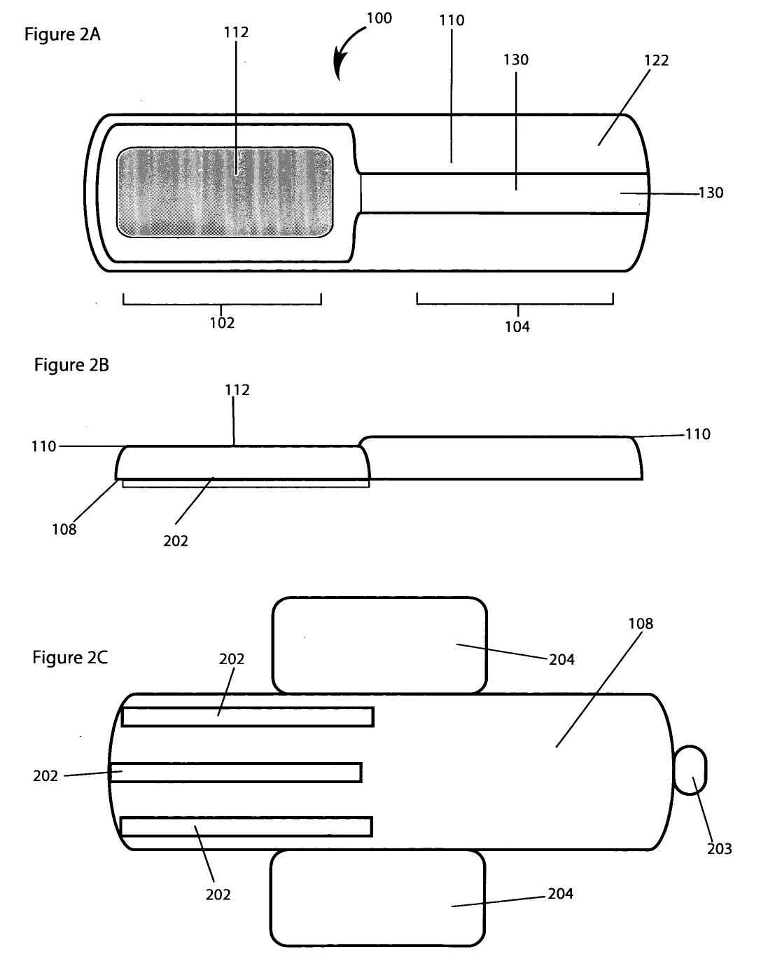

Liquid pervious surface 112 of proximal surface 110 shown in FIGS. 1A-C is shown covering the entire proximal surface. Liquid pervious surface 112, however, may cover only one or more regions of proximal surface 110. For example, FIG. 2A shows a fluid pervious region 112 located in the central part of proximal surface 110 of pad region 102. In another embodiment, the fluid pervious surface 112 covers one or more of the central portion of proximal surface 110 of absorbent article 100. The remainder of proximal surface 110 of the pad region 102 may comprise a material having other properties, for example, without limitation, a material which is fluid impervious. In one embodiment, one or more regions of proximal layer 110 comprise more than one layer of liquid pervious material.

Although the portion of pad region 102 of absorbent article 100 is shown as substantially flat in the figures, it may be of any shape. In particular, the pad region 102 may be a shape that conforms to the shape of a wearer's body (for example, without limitation, the interlabial region of a user). In some embodiments, the proximal portion of pad region 102 (the body-facing side of the pad region) has a three-dimensional structure. In another embodiment, pad region 102 has a ridge running longitudinally down the center of the pad region, (typically, substantially perpendicular to line B-B′ in FIG. 1A). In a further embodiment, the proximal surface of pad region 102 includes a tubular structure. In some embodiments, this tubular structure extends into gluteal region 104. In some embodiments, the pad region is conformal to a user's body. In a particular embodiment, the shape of pad region 102 may be modified by the user applying absorbent article 100.

Pad region 102 of FIG. 1A is shown as substantially rectangular along its outer perimeter. Pad region 102, however, may comprise any shape effective to be comfortably worn by a user and able to prevent leakage of emitted bodily fluids. In an embodiment, pad region 102 is oval-shaped. In another embodiment, pad region 102 is hourglass-shaped. In yet another embodiment, pad region 102 is triangular shaped with the triangle pointing posteriorly.

FIG. 1B shows a cross-sectional view of pad region 102 taken along line B-B′ of FIG. 1A. Pad region 102 as shown has a proximal liquid pervious surface 112, a distal surface (or garment-facing surface) 108, and an absorbent core 106 located between the proximal and distal surfaces, 108, 110. Distal surface 108 comprises a liquid impervious surface so that fluid absorbed by absorbent core 106 is retained in the device.

The lateral sides 120 of pad region 102 shown in FIG. 1B may connect to the proximal and distal surfaces, enclosing the pad region within. In one embodiment, the sides comprise the same liquid impervious material as the distal surface. Lateral sides 120 may be a defined region as shown in FIG. 1B, or may be a minimal union between the proximal and distal surfaces, 108, 110. In one embodiment, the lateral sides 120 are continuous with the distal surface 108. In another embodiment, the sides are a seam formed between the two materials comprising the proximal and the distal surfaces 108, 110. In yet another embodiment, the sides further comprise an elastic material.

The liquid impervious material of distal surface 108 of absorbent article 100 may be of any suitable material capable of preventing fluid from passing out of device 100. Preferably, the liquid impervious material is flexible enough to allow pad region 102 of absorbent article 100 to conform to a user's body. Also, the liquid impervious material is strong enough to maintain the structural integrity of the device (for example, without limitation, prevent tearing of the device and/or loss of the absorbent core). For example, the liquid impervious material may comprise a material similar to those of the liquid pervious material (only, for example, without the pores which allow fluid passage). Examples of liquid impervious materials include woven and non-woven materials; polymeric materials such as formed thermoplastic films, plastic films, hydroformed thermoplastic films. In one embodiment, the liquid impervious material is a hydrophobic coating. In another embodiment, the liquid impervious material of the distal portion of the device is a polyethylene film. In yet another embodiment, the distal layer comprises more than one layer of liquid impervious material.

Pad region 102 of absorbent article 100 is connected to gluteal region 104. In particular, absorbent core 106 of pad region 102 is in fluid connection with absorbent core 106 of gluteal region 104. In an embodiment, absorbent core 106 of pad region 102 is part of the same structure as absorbent core 106 of gluteal region 104. In another embodiment, proximal surface 110 of pad region 102 is part of the same structure as outer proximal surface 110 of gluteal region 106. See FIGS. 1A-1C. In yet another embodiment, distal surface 108 of pad region 102 is part of the same structure as at least a portion of gluteal region 108, 104. See FIGS. 1A-1C.

Fasteners

FIG. 2B shows a lateral side view of absorbent article 100 of FIG. 2A. FIG. 2C shows a distal view of absorbent article 100 of FIG. 2A. In this particular embodiment, three fasteners 202 are shown running approximately halfway along distal surface 108 of absorbent article 100, beneath pad region 102. Fasteners may be used to attach the absorbent article to garments (for example, without limitation, undergarments) worn by a user. The fasteners may be of any type sufficient to secure one or more portions of the absorbent article to one or more portions of a garment. In one embodiment, the fastener is an adhesive. In other embodiments, the number and size of the adhesive fasteners can vary. Other examples of fasteners include mechanical fasteners (for example, without limitation, pins, snaps, buttons, etc).

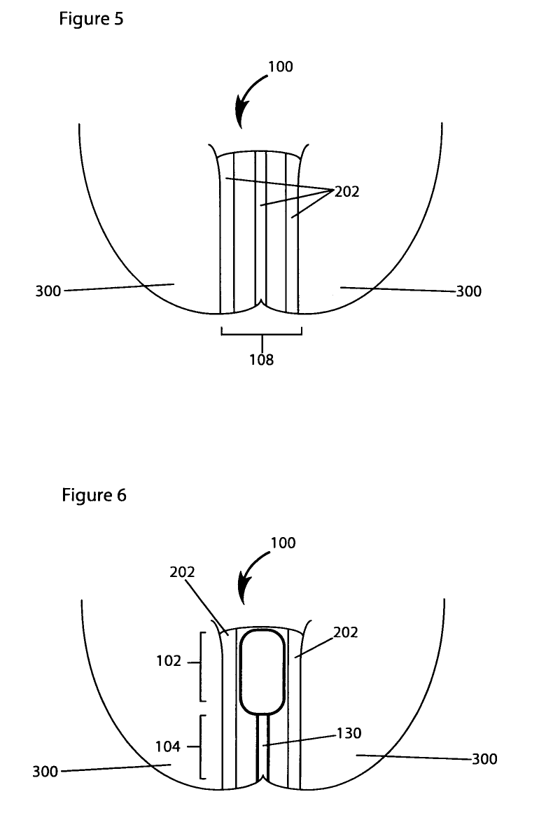

Although FIG. 2C shows three fasteners 202 running longitudinally along distal surface 108 of pad region 102, different configurations (including different numbers and lengths of fasteners) are also included as part of this description. One, two, three or more than three fasteners may be included as part of the device. Fasteners may also be of virtually any size. In FIG. 2C each fastener 202 has a relatively narrow width compared with the width of the pad region 102 and a length approximately the same as pad region 102. In another embodiment, at least one fastener 202 extends the entire length of the device. See FIGS. 5 and 6. In yet another embodiment, at least one fastener extends less than a few centimeters in length. In a further embodiment, at least one fastener is a double-sided adhesive having a removable strip to expose the adhesive so that it will attach to a garment (for example, without limitation, an undergarment).

Fasteners may also be used to secure one or more regions of the absorbent article to one or more regions of the user. In one embodiment, fastener 203 secures the end of gluteal region 104 of absorbent article 100 to the skin of the user. See FIG. 2C. In this case, fastener 203 is attached to the end of gluteal region 104, and is configured to attach to the user by an adhesive. In one embodiment, this adhesive is any biocompatible adhesive that may be used in contact with the user's skin as known to those skilled in the art. For example, but not limited to, the adhesive may be any medically acceptable adhesive as commonly used for bandages or other skin adhering devices. In another embodiment, the fastener is attached to absorbent article 100 posteriorly via extension of bib 122. Fastener 203 may be attached to bib 122 on either the proximal user-contacting surface, the distal garment-contacting surface, or both. See FIG. 2C.

The fasteners may also comprise “wings” 204 at the lateral sides of the absorbent article 100, which are configured to fold over an undergarment to secure the pad to the undergarment by adhesive means known to those skilled in the art. The adhering means is typically located on the distal surface (for example, without limitation, the same surface as fasteners 202). Wings 204 would wrap around and adhere to the outer distal surface of the user's garment (for example, without limitation, typically the non-body facing surface of the crotch of a panty). In another embodiment, the absorbent article includes wings 204. In a particular embodiment, wings 204 are contiguous with distal surface 108 of absorbent article 100. In other embodiments, the proximal side of wings 204 have a fluid pervious surface 110 contiguous with pad region 102, gluteal region 104, or both regions. In another embodiment, the distal garment facing side of the wings comprise a fluid impervious surface that may or may not be contiguous of fluid impervious layer 108 of pad region 102, gluteal region 104, or both. In yet another embodiment, wings 204 may also comprise an absorbent core between the fluid pervious and fluid impervious surfaces. The absorbent core of the wings may or may not be contiguous with absorbent cores 106 of pad region 102, gluteal region 104, or both. In another embodiment, the absorbent article does not include wings.

Gluteal Region

Gluteal region 104 comprises an absorbent core 106, which is in fluid connection with absorbent core 106 of pad region 102. In operation, when absorbent article 100 is worn by a user, gluteal region 104 extends posteriorly from pad region 102, and fits in the gluteal region of the user. In one embodiment, gluteal region 104 fits between buttocks 300 of the user. See FIG. 6. In one embodiment, gluteal region 104 extends towards the coccyx region. Thus, gluteal region 104 may help anchor and support the absorbent article during use, as well as absorbing fluid (for example, without limitation, menstrual fluid) from pad region 102.

Gluteal region 104 is configured to include a gluteal insertion region 130. Gluteal insertion region 130 comprises a pre-formed member that may be inserted between buttocks 300 of the user. In general, gluteal region 104 is non-corrugated, meaning that gluteal region 104 has a preformed gluteal insertion region 130, and does not rely upon the user to apply force to form gluteal insertion region 130 (for example, without limitation, to fold or compress the gluteal region to form the gluteal insertion region). Thus, gluteal region 104 generally comprises a longitudinal member, such as, but not limited to, a tubular member, cylinder, or ridge.

FIG. 1A shows a view of one embodiment of gluteal region 104 described herein. Gluteal region 104 is connected to the pad region at the posterior end of pad region 102 (to the right of the pad region in FIG. 1A). FIG. 1C shows a cross-section of gluteal region 102 of FIG. 1A. In FIG. 1C, gluteal region 102 comprises a gluteal insert region 130 having an absorbent core region 106 and a bib region 122. In cross-section, bib region 122 lies on either side of at least a portion of gluteal insert region 130. In another embodiment, bib 122 can extend more posteriorly beyond the posterior edge of gluteal region 104. In FIGS. 1 and 2, bib region 122 extends the same length as the absorbent core region 106. Bib region 122 may help prevent moisture from leaking onto user's clothing by projecting posteriorly from pad region 102.

Absorbent core 106 of gluteal insert region 130 shown in FIGS. 1A and 1C is covered in at least one covering material. As shown, the absorbent core has a proximal surface, continuous with proximal surface 110 of pad region 102, and a distal surface which is continuous with the distal surface 108 of pad region 102. Thus, the proximal surface may comprise a liquid pervious material, and the distal surface may comprise a liquid impervious material. Both the proximal and distal surfaces of the bib region 122 may comprise a liquid impervious material.

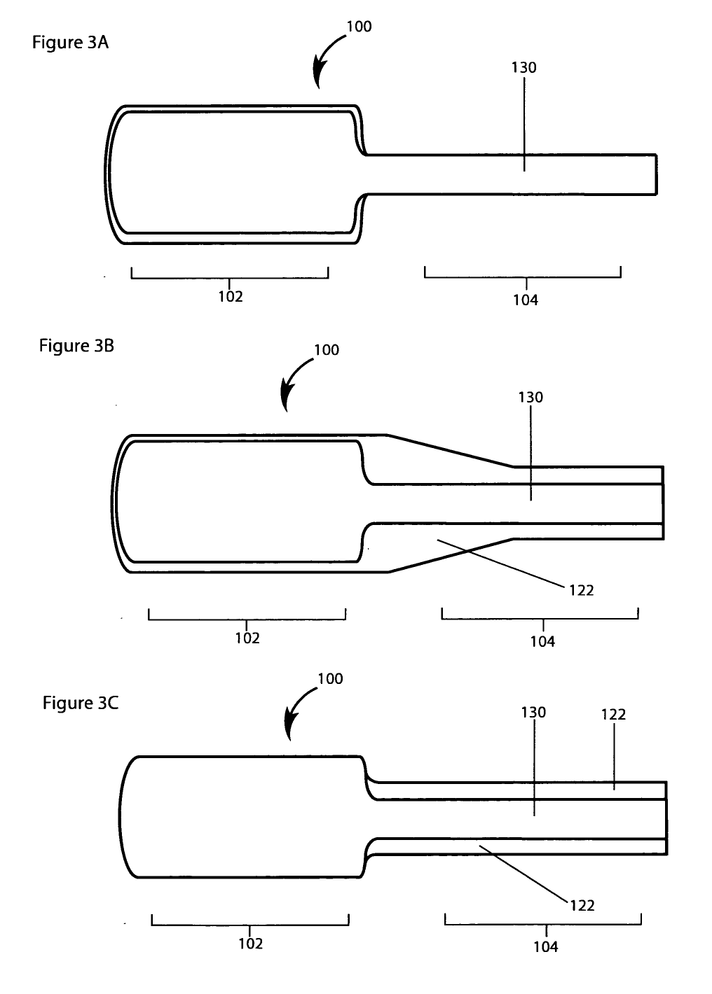

Bib region 122 may also comprise different shapes and materials. FIG. 3A is a proximal view of one embodiment of an absorbent article 100, including a gluteal region 104 that does not include any bib region. Thus, pad region 102 is continuous with gluteal region 104. FIG. 3B is a proximal view of another embodiment of absorbent article 100 having a gluteal region 104 comprising a tapered bib region 122 extending from pad region 102. In FIG. 3B, gluteal insert region 130 connects to bib region 122 along at least the distal side of the bib region, so that gluteal insert region 130 projects above the proximal surface of bib region 122, and can be inserted between buttocks 300 of a user. See also FIGS. 5 and 6.

Another variation of bib region 122 of a gluteal region 104 is shown in FIG. 3C, showing a bib region 122, which extends laterally from gluteal insert region 130 a distance less than the width of the absorbent pad. Bib region 122 may have virtually any shape or combination of shapes. Bib 122 may also extend posteriorly, and alternatively have a fastener 203 (see FIG. 2C), which attaches either to the user, the user's garment, or both. Further, bib region 122 may also comprise an absorbent material, which may also be in fluid contact with absorbent core 106.

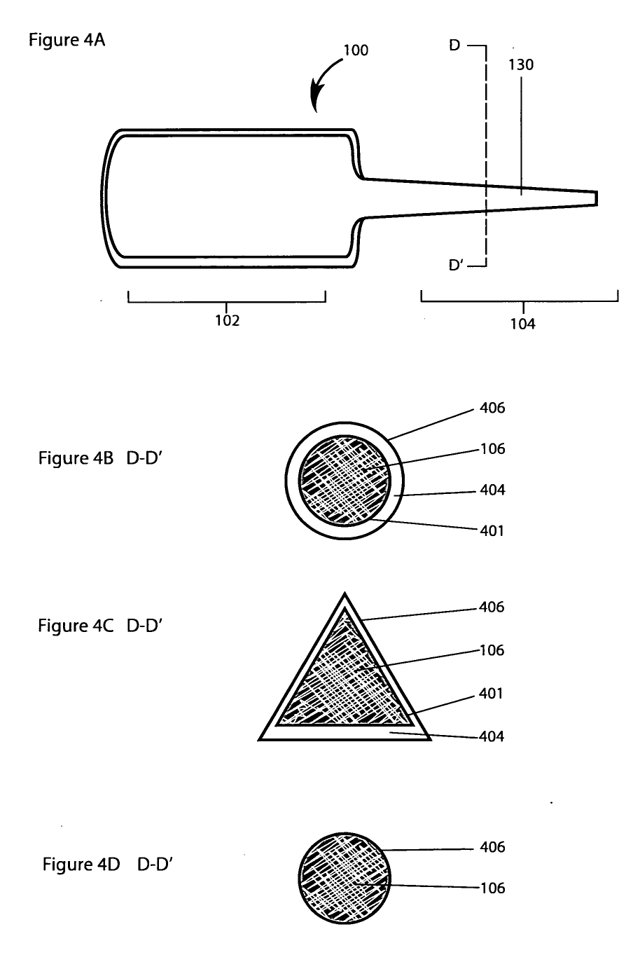

Gluteal insert region 130 may also be of different shapes and sizes. FIG. 4A shows a proximal view of an absorbent article having a tapered gluteal insert region 130. The tapered gluteal insert region 130 is shown as widest where gluteal region 104 is attached to pad region 102, becoming narrower as the gluteal region 104 extends posteriorly. Gluteal region 104, however, may also vary across its length, including narrowing and then getting wider again. In one embodiment, gluteal region 104 starts narrow and gets wider as it extends posteriorly.

FIGS. 4B to 4D show variations of cross-sectional views of gluteal insert region 130 of an absorbent article 100. In FIG. 4B, the cross-section of gluteal region 104 is circular. Gluteal region 104 in FIG. 4 comprises a gluteal insert region 130 having an absorbent core 106. In FIG. 4B, at least a portion of the absorbent core 106 is surrounded by an outer region 404. In some embodiments, this outer region 404 is isolated from absorbent core region 106. In another embodiment, absorbent core 106 is surrounded by an inner gluteal region surface 401 that comprises a liquid impervious surface preventing fluid in absorbent core 106 of gluteal region 104 from contacting the user or the user's clothing. In other embodiments, an outer absorbent region 404 surrounds absorbent core 106 and comprises an outer absorbent region 404 and/or an outer gluteal region surface 406. Outer gluteal region surface 406 may comprise a liquid pervious material, thereby allowing liquids (for example, without limitation, sweat, blood, etc.) in this region to be absorbed by gluteal region 104. Outer absorbent region 404 may be comprised of a material similar to absorbent core material 106.

In some embodiments, outer region 404 is at least partly in fluid contact with absorbent core 106. Thus, a portion (or all) of inner gluteal region surface 401 may comprise a liquid pervious material, or may be absent. The different components of the gluteal region (for example, without limitation, absorbent core 106, inner gluteal region surface 401, outer absorbent region 404, and/or outer gluteal region surface 406) may extend through the entire length of gluteal region 104, or may extend only partially. Inner 401 and outer 406 gluteal region surfaces can comprise either liquid pervious or liquid impervious materials; examples of such materials were described above.

FIG. 4C shows another embodiment of gluteal region 104 in cross-section. In FIG. 4C, the gluteal insert region has an overall triangular shape. Absorbent core 106 is also shown as triangular, however, it could be any shape sufficient to fit within the gluteal region of the user. The embodiment of gluteal region 104 shown in FIG. 4C also includes an absorbent core 106, an inner gluteal region surface 401, an outer gluteal region 404, and an outer gluteal surface 406. The cross-sectional shape of gluteal region 104 may comprise any shape, for example, without limitation, circular, triangular, rectangular, etc.

FIG. 4D shows an embodiment of gluteal region 104 comprising an absorbent core 106 and an outer gluteal region surface 406. In FIG. 4D, outer gluteal region surface 406 may be either fluid pervious or fluid impervious. The absorbent core 106 of gluteal region 104 may thus act as a reservoir to absorb excess fluid from pad region 102 in addition to helping secure absorbent article 100.

Gluteal region 104 is configured to have gluteal insert region 130 fit between buttocks 300 of the user. See FIGS. 5 and 6. In one embodiment, gluteal insert region 130 of gluteal region 104 is tucked between buttocks 300. See FIGS. 5 and 6. In another embodiment, gluteal region 104 comprises a compressible material. In yet another embodiment, gluteal region 104 comprises an incompressible material. Gluteal region 104 may also be flexible, so as to more comfortably fit gluteal insert region 130 between buttocks 300 of the user. In a further embodiment, gluteal region 104 is configured from flexible materials allowing gluteal region 104 to easily bend.

Absorbent core 106 of gluteal region 104 may be configured to absorb fluid from pad region 102. In a particular embodiment, absorbent core 106 of gluteal region 104 contacts absorbent core 106 of pad region 102. In one embodiment, absorbent core 106 absorbs fluid from pad region 102 by wicking. Absorbent core 106 of gluteal region 104 may act as a reservoir, storing excess fluid from pad region 102 and keeping pad region 102 relatively dry, enhancing the user's comfort.

Absorbent Core

Absorbent core 106 may comprise any appropriate material capable of absorbing one or more of the fluids to be absorbed by the device. The absorbent core may comprise any mixture of such materials; further, different regions of the device may comprise different absorbent core materials that are in fluid contact with each other. For example, absorbent core 106 of pad region 102 may comprise a different absorbent core material than absorbent core 106 of gluteal region 104.

In some embodiments, the absorbent core material is any of the liquid-absorbent materials commonly used in sanitary napkins and other absorbent articles such as comminuted wood pulp (for example, without limitation, airfelt), creped cellulose wadding; meltblown polymers (for example, without limitation, coform); chemically modified cellulose fibers (for example, without limitation, crosslinked, stiffened, etc.); synthetic fibers (for example, without limitation, crimped polyester fibers); peat moss; tissue (for example, without limitation, tissue laminates); absorbent foams; sponge material (natural and artificial); absorbent or superabsorbent polymeric material (for example, without limitation, supersoft polymers, dried hydrogels); natural or synthetic fibers (for example, without limitation, cotton); woven and non-woven material; absorbent gelling materials; or any combination or mixture of these. The absorbent material may also comprise (or partly comprise) less absorbent, or non-absorbent, materials such as polyurethane foam, which can be formed into absorbent structures when combined with absorbent material and which can provide wicking and therefore even dispersion of fluids throughout the absorbent core.

The absorbent material may be formed into structures. In one embodiment, the absorbent material of the pad is shaped to have the appropriate thickness of pad region 102 (when added to the distal and proximal surfaces). In another embodiment, the absorbent material is a foam (for example, without limitation, high internal phase emulsion or HIPE foam). In yet another embodiment, the absorbent material comprises pellets. Pellets of absorbent material may be connected (for example, without limitation, as in a matrix or webbing), loose, or some combination thereof. Pellets of absorbent material may be of any appropriate size and/or shape, or range of sizes and/or shapes.

Overall Structure

The absorbent article 100 may be greater than 12 inches in length in order to cover the genitourinary region of the user and extend between (or over) the buttocks 300 towards the coccyx or tailbone region. The size of absorbent article 100 may be selected by the designer so that the absorbent article is configured to fit one of a variety of body shapes and sizes. In an embodiment, the size of the absorbent article 100 may be selected based on a range of body measurements taken from a user. Examples of measurements, which may be correlated to body sizes, include waist diameter, hip diameter, pelvic size, etc. In another embodiment, the overall length of absorbent article 100 may vary from between about 12 inches to about 22 inches. In other embodiments, the length of absorbent article 100 may vary from between about 13 inches to about 21 inches. In yet another embodiment, the length of absorbent article 100 may vary from between about 14 inches to about 20 inches. As used herein, length may be measured from a “flattened” absorbent article 100, although the article itself may be curved or compressed. It should be recognized by those of skill in the art that a large number of design variations of the described structures are possible.

The width of absorbent article 100 may also vary. In FIGS. 1 and 2, absorbent article 100 is shown as having a constant width, however the width may vary over the length of absorbent article 100. Further, absorbent article 100 may be differently compressible over its width, in order to better conform to the body of a user. In an embodiment, the width may vary from between about 2 inches to about 4 inches in width. In another embodiment, the width may vary from between about 2 inches to about 3 inches in width. In yet another embodiment, the width is about 2 and ½ inches in width.

Further, the relative contribution of pad region 102 and gluteal region 104 may vary. In a particular embodiment, pad region 102 is between about 3 inches and about 10 inches long and gluteal region 104 is between about 3 inches and about 18 inches long. In another embodiment, pad region 102 is between about 5 inches and about 9 inches long and gluteal region 104 is between about 5 inches and about 17 inches long. In yet another embodiment, gluteal region 104 is between about 6 inches and about 8 inches long, and gluteal region 104 is between about 8 inches and about 16 inches long. Further, the size of pad region 102 and gluteal region 104 may be adjusted based on the fit and comfort of the user.

The thickness of absorbent core 106 (and thus the thickness of pad region 102 and/or gluteal region 104) may also be adjusted. The thickness of pad region 102 may be uniform over the pad region 102, or the thickness may vary over pad region 102.

Table 1 gives examples of sizes of absorbent articles 100 based on theoretical measurements taken from potential users. These examples are for illustration only, and should not be construed to limit the device claims.

TABLE 1
Examples of sizes

| | Waist Size | Hip Size | Absorbent | | Pad |
|---|---|---|---|---|---|
| | of user | of user | Article Length | Pad Width | Thickness |
| Size | (inches) | (inches) | (inches) | (inches) | (cm) |
| Small | 28½ to | 38½ to | 14 | 2½ | 1 |
| | 29½ | 39½ | | | |
| Medium | 30 to | 40½ to | 16 | 2½ | 1 |
| | 33½ | 43½ | | | |
| Large | 34 to | 44 to | 18 | 2½ | 1 |
| | 37½ | 47½ | | | |
| X-Large | 38 to | 48 to | 20 | 2½ | 1 |
| | 41½ | 51½ | | | |

Additional Components

The device may also incorporate additional features such as chemical treatments (for example, without limitation, deodorants, antibacterial agents, as are known in the feminine hygiene industry). For example, absorbent article 100 may be sterilized, and packaged in a sterile, compact packaging. In one embodiment, absorbent article 100 is foldable along at least one axis to facilitate storage and/or packaging. In another embodiment, absorbent article 100 is made of a material that may be readily sterilized.

In a particular embodiment, absorbent article 100 incorporates a deodorant into at least a region of the material comprising the absorbent article. In another embodiment, absorbent article 100 incorporates an antibacterial agent into one or more of the materials comprising the absorbent article. For example, the absorbent material of the absorbent core may further comprise and antibacterial or deodorant agent.

In use, a user may apply absorbent article 100 to absorb body secretions. FIG. 5 shows an example of an absorbent article as described herein being worn by a user. Absorbent article 100 is fits between the legs. Pad region 102 fits over the user's genitourinary region, and gluteal region 104 fits between the user's buttocks 300. In FIG. 5, the distal surface of the absorbent article 100 is shown, extending posteriorly towards the tailbone (coccyx). Three fasteners 202 (adhesive strips) are shown extending the length of the absorbent article; the fasteners 202 may be attached to the users clothing.

FIG. 6 shows a cut-away embodiment of absorbent article 100 shown in FIG. 5, exposing some of the details of absorbent article 100. The outline of pad region 102 is shown surrounding the genitourinary region. Gluteal insert region 130 of gluteal region 104 is shown tucked between the user's buttocks 300. In operation, liquid is absorbed by pad region 102 and wicked into gluteal insert region 130 because absorbent core 106 of gluteal insert region 130 is in fluid communication with pad region 102.

I claim:

1. An absorbent article for absorbing a bodily fluid comprising:
a pad region comprising a first absorbent core, a liquid pervious surface, and a liquid impervious surface, wherein the absorbent core of the pad region is positioned between the liquid pervious surface and the liquid impervious surface;
a non-corrugated, conformable gluteal region comprising a second absorbent core in fluid connection with the first absorbent core, wherein the gluteal region is configured to secure at least a portion of the absorbent article between a user's buttocks, to wick fluid from the pad region; and
the absorbent article extends at least about 12 inches in length.

2. The absorbent article of claim 1, wherein at least the first absorbent core is a non-woven material.

3. The absorbent article of claim 2, wherein at least the first absorbent core is cotton.

4. The absorbent article of claim 1, wherein the absorbent article further comprises at least one fastener adapted to attach to a garment.

5. The absorbent article of claim 4, wherein the fastener comprises adhesive tape.

6. The absorbent article of claim 4, wherein the fastener comprises wings.

7. The absorbent article of claim 4, wherein the fastener does not include wings.

8. The absorbent article of claim 4, wherein at least one fastener is located on the liquid impervious surface of the pad region.

9. The absorbent article of claim 8, wherein the fastener is attached to the gluteal region.

10. The absorbent article of claim 9, wherein the fastener adheres to the skin of the user.

11. The absorbent article of claim 1, further comprising a deodorant.

12. The absorbent article of claim 1, further comprising an antibacterial agent.

13. The absorbent article of claim 1, wherein the gluteal region further comprises a flexible gluteal insert region.

14. The absorbent article of claim 13, wherein the gluteal insert region is a cylinder.

15. The absorbent article of claim 1, wherein the gluteal region is tapered.

16. The absorbent article of claim 1, wherein the width of the gluteal region is narrower than the width of the pad region.

17. The absorbent article of claim 1, wherein the absorbent article substantially conforms to a female perineal body region and is configured to absorb menstrual fluid.

18. The absorbent article of claim 1, wherein the absorbent article is foldable.

19. An absorbent article for absorbing a bodily fluid from a user comprises:
a pad region comprising a liquid pervious surface, a liquid impervious surface and a first region of an absorbent core positioned between the liquid pervious and liquid impervious surfaces; and
a gluteal region comprising a second region of the absorbent core, wherein at least a portion of the gluteal region provides positional stability for the absorbent article when worn by a user;
wherein the gluteal region further comprises a gluteal insert region; and
wherein the absorbent article extends at least about 12 inches in length.

20. An absorbent article for absorbing a bodily fluid from a user comprises:
a pad region comprising a liquid pervious surface, a liquid impervious surface and a first region of an absorbent core positioned between the liquid pervious and liquid impervious surfaces;
a gluteal region comprising a second region of the absorbent core, wherein at least a portion of the gluteal region provides positional stability for the absorbent article when worn by a user; and
wherein the gluteal region further comprises a flexible gluteal insert region; and wherein the gluteal region is configured to wick fluid from the pad region; and
further wherein the absorbent article extends at least about 12 inches in length.
